(19) United States
(12) Patent Application Publication
(10) Pub. No.: US 2007/0231274 A1
(43) Pub. Date: Oct. 4, 2007

(54) Mouthwash and Method of Using Same for the Treatment of Mucositis or Stomatitis

(75) Inventor: Deepak Bhasin, Richmond Hill (CA)

Correspondence Address: BLAKE, CASSELS & GRAYDON LLP, BOX 25, COMMERCE COURT WEST, 199 BAY STREET, SUITE 2800, TORONTO, ON, M5L 1A9, US

(73) Assignee: Myrex Pharmaceuticals Inc., Toronto (CA)

(21) Appl. No.: 11/692,737

(22) Filed: Mar. 28, 2007

Related U.S. Application Data
(60) Provisional application No. 60/786,376, filed on Mar. 28, 2006.

(51) Int. Cl. A61K 8/73; A61K 31/7048; A61K 31/573

(52) U.S. Cl. 424/49; 514/29; 514/171

(57) ABSTRACT

An oral rinse formulation for the treatment or prophylaxis of mucositis or stomatitis comprises one or more of the following components in combination with pharmaceutically acceptable excipients or additives: a) at least one corticosteroid; b) at least one anti-histamine; c) at least one topical anaesthetic; d) at least one antibiotic agent; and, e) at least one anti-fungal antibiotic agent. A method of treating or preventing mucositis or stomatitis using such formulation is provided.

### CROSS REFERENCE TO PRIOR APPLICATIONS

This application claims priority from U.S. application No. 60/786,376, filed on Mar. 28, 2006, the entire contents of which are incorporated herein by reference.

### BACKGROUND OF THE INVENTION

The present invention relates to oral hygiene and in particular, to mouthwashes for the treatment and prophylaxis of mucositis and stomatitis of the oral cavity and to a method of using such a mouthwash.

Mouthwashes are typically used as part of an oral hygiene regime which may also include brushing and flossing to maintain a satisfactory level of oral hygiene. Such mouthwashes are generally classified as cosmetic, therapeutic or a combination of the two. Cosmetic rinses are commercial over-the-counter products that help remove oral debris before or after brushing, temporarily suppress bad breath, diminish bacteria in the mouth and refresh the mouth with a pleasant taste. Therapeutic rinses often have the benefits of their cosmetic counterpart, but also contain an added active ingredient, for example chlorhexidine that helps protect against some oral diseases such as gingivitis. Typically, such mouthwashes are alcohol-based.

However, available mouthwashes are unsuitable for hospital patients presenting with oral complications as a result of, for example, infection or other lesions as a consequence of chemotherapy or radiotherapy for the treatment of neoplastic disease. Such oral complications may include mucositis or stomatitis. Mucositis or stomatitis presents in approximately 40% of patients with neoplastic disease.

Examples of chemotherapeutic drugs that frequently cause mucositis and/or stomatitis include alkylating agents, for example melphalan and busulphan; antimetabolites, for example, cytarabine, floxuridine, 5-fluorouracil, mercaptopurine, methotrexate, and thioguanine and cytotoxic drugs, for example, bleomycin, actinomycin D, daunorubicin, cisplatin, etoposide, mytomycin, vinblastine and vincristine.

The terms mucositis and stomatitis are often used interchangeably but may include some general distinctions. Mucositis describes a toxic inflammatory reaction affecting the gastrointestinal tract, which may result from exposure to chemotherapeutic agents or ionizing radiation. Mucositis typically manifests as an erythematous burn-like lesion, or as random focal-to-diffuse lesions. Stomatitis refers to an inflammatory reaction affecting the oral mucosa, with or without ulceration, that may be caused or intensified by pharmacological, particularly chemotherapeutic treatments or by radiotherapy. Stomatitis can range from mild to severe; a patient presenting with the severe stomatitis is unable to take anything by mouth.

Current oral cleaning care in such patients may include gently cleaning the mouth, moisturizing the lips and mouth, and relieving pain and swelling. A soft toothbrush cleans teeth well and gently. Cleansing agents can include “salt and soda” (half a teaspoon of salt and two tablespoons of sodium bicarbonate in 32 ounces of warm water), saline, sterile water or sodium bicarbonate (one teaspoon in 8 ounces of water). Hydrogen peroxide diluted in equal amounts of water or weak salt water can be used in some cases. Mostly, physicians have resorted to these rather limited, temporary relief options.

It is therefore extremely important that mucositis and stomatitis be prevented whenever possible or at the very least that they be reduced in their severity and possible complications. Currently there are a number of intervening therapies to choose from. For example, The Joanna Briggs Institute of The Royal Adelaide Hospital has provided some guidance on the treatment of mucositis. However, there is no high quality synthesis of research evidence for these intervention therapies (www.joannabriggs.edu.au/best_practice/bp5.php, 19 Feb. 2004).

Of particular interest to the current application are the variety of mouthwashes previously used having mixed actions against mucositis and stomatitis. Such mouthwashes typically include benzydamine hydrochloride, corticosteroids and chamomile (www.joannabriggs.edu.au/best_practise/bp5.php)

Formulations such as those disclosed in U.S. Pat. No. 5,635,489; U.S. Pat. No. 4,961,926 and U.S. Pat. No. 5,102,870 describe the use of growth factors and stimulation factors such as granulocyte-macrophage colony stimulating factor and granulocyte-colony stimulating factor.

Further attempts at treatment and or prophylaxis of mucositis and stomatitis include the use of nucleoside derivatives which can be formulated into lozenges or mouthwashes and the like to coat the oral cavity or other mucosal areas such as disclosed in U.S. Patent Publication No. 2003/0236217 A1.

Other formulations include tetracycline as disclosed in U.S. Pat. No. 6,946,118; hyaluronic acid, glycyrrhetinic acid and polyvinylpyrrolidone in mixture with excipients and adjuvants as disclosed in U.S. Pat. No. 6,828,308; the use of an anti-microbial peptide, preferably protegrin applied topically which has broad spectrum anti-microbial activity, good stability and adheres well to all mucosa as disclosed in U.S. Pat. No. 6,025,326; the use of glutamine as disclosed in U.S. Pat. No. 5,545,668 and the use of triclosan as disclosed in U.S. Pat. No. 5,945,089

Despite the widespread recognition that mucositis is a serious problem, no effective treatment currently exists and, more often than not, any level of patient care is typically palliative. In addition to the lack of effective treatments for mucositis, physicians are unsure of the exact mechanism by which the ulcerations occur in mucositis. It is known that the initial exposure to a chemotherapeutic agent causes a release of cytokines from the epithelial tissues. Subsequently, mitosis is disturbed in the epithelia. Finally, there are alterations in the bacterial flora of the oral cavity. It is unknown which of these occurrences, if any, is responsible for the mucosal damage. Despite the fact that significant quantities or inflammatory mediators are released by the epithelium, conventional anti-inflammatory agents have been unsuccessful in human efficacy studies of the disease.

Hitherto, the available mouthwashes are effective at treating only one aspect of mucositis and other oral complications and not treating the complete range of symptoms that present in patients.

### SUMMARY OF THE INVENTION

In one aspect, the present invention provides an oral rinse formulation comprising one or more of the following components in combination with pharmaceutically acceptable excipients or additives:

a) at least one corticosteroid;

b) at least one anti-histamine;

c) at least one topical anaesthetic;

d) at least one antibiotic agent; and,

e) at least one anti-fungal antibiotic agent.

In a further aspect, the invention provides a method of treating or preventing mucositis or stomatitis comprising applying to a patient the above mentioned oral rinse formulation.

### DETAILED DESCRIPTION OF THE INVENTION

Accordingly, the present invention provides, in one embodiment, a mouthwash comprising one or more of the following active ingredients:
at least one corticosteroid;
at least one anti-histamine;
at least one topical anaesthetic;
at least one antibiotic agent; and,
at least one anti-fungal antibiotic agent.

Preferably, the mouthwash in accordance with the present invention is used in the treatment and/or prophylaxis of oral conditions such as mucositis and/or stomatitis.

Preferably the mouthwash in accordance with the present invention further comprises water and pharmaceutically acceptable excipients or additives such as:
one or more oils, such as an oil selected from the group comprising anethole, anisole, camphor, methyl salicylate, vanillin, eugenol, furaneol, linalool, menthol, thymol, cinnamaidehyde, citral, methyl butanoate, pentylbutanoate, pentylpentanoate, tea tree oil, peppermint oil, spearmint oil, pineapplemint oil and eucalyptus oil;
sweetening agents, for example sorbitol;
thickening agents, such as xanthan gum, carrageenan or carbomer;
preservative agents, such as sodium benzoate;
water;
emulsifiers, such as polysorbate 20;
and/or at least one antacid such as aluminium or magnesium hydroxide.

It will be appreciated by persons skilled in the art that the above list of excipients and/or additives is provided merely by way of example and that various other such components may be used in the formulation of the present invention.

Oral mucositis results in specific damage that includes the shedding of the mucosal lining of the mouth (desquamation), ulceration and atrophy. Aluminum and magnesium hydroxide antacids coat and protect the oral cavity from damage and provide for re-growth of normal oral tissue.

The corticosteroid component provides anti-inflammatory action directly on the oral mucosa and works to limit the inflammatory response associated with mucositis. Preferably, the corticosteroid is Dexamethasone.

In patients being treated with chemotherapy and radiation, it is important to take steps to prevent bacterial infection within the oral cavity. The antibiotic component of the mouthwash formulation of the invention may be of the macrolide type and may be selected from the group consisting of Erythromycin, Azithromycin, Clarithromycin, Dirithromycin, Roxithromycin Carbomycin A, Josamycin, Kitasamycin, Oleandomycin, Spiramycin, Troleandomycin, Tylosin, Cethromycin, Ansamycin and Telithromycin. In one aspect the antibiotic is Erythromycin. Erythromycin is used to achieve this antibacterial environment. Once ulcerations develop inside the mouth, local oral bacteria colonize the wound and release cell wall products into the mucosa, resulting in an amplification of a tissue destructive cycle. The antibiotic component, i.e. Erythromycin, limits such bacterial colonization. As will be understood, limiting the extent of bacterial infection would promote healing of the affected tissues.

Alternatively, the antibiotic may be any of the following, alone or in combination:
an aminoglycoside, for example, Amikacin, Gentamicin, Kanamycin, Neomycin, Netilmicin, Streptomycin, and Tobramycin;
a carbacephem, for example, Loracarbef;
a carbapenem, for example, Ertapenem, Imipenem/Cilastatin, and Meropenem;
a cephalosporin (First generation), for example, Cefadroxil, Cefazolin, Cephalexin;
a cephalosporin (Second generation), for example, Cefaclor, Cefamandole, Cefoxitin, Cefprozil, and Cefuroxime;
a cephalosporin (Third generation), for example, Cefixime, Cefdinir, Cefditoren, Cefoperazone, Cefotaxime, Cefpodoxime, Ceftazidime, Ceftibuten, Ceftizoxime, and Ceftriaxone;
a cephalosporin (Fourth generation), for example, Cefepime;
a glycopeptide, for example, Teicoplanin, Vancomycin;
a penicillin, for example, Amoxicillin, Ampicillin, Aziocillin, Carbenicillin, Cloxacillin, Dicloxacillin, Flucloxacillin, Mezlocillin, Nafcillin, Penicillin, Piperacillin and Ticarcillin;
a polypeptide, for example, Bacitracin, Colistin, and Polymyxin B;
a quinolone, for example, Ciprofloxacin, Enoxacin, Gatifloxacin, Levofloxacin, Lomefloxacin, Moxifloxacin, Norfloxacin, Ofloxacin, and Trovafloxacin;
a sulfonamide, for example, Mafenide, Prontosil, Sulfacetamide, Sulfamethizole, Sulfanilimide, Sulfasalazine, Sulfisoxazole, Trimethoprim, and Trimethoprim-Sulfamethoxazole;
a tetracycline, for example, Demeclocycline, Doxycycline, Minocycline, and Oxytetracycline, Tetracycline, and
another antibiotic, for example, Chloramphenicol, Clindamycin, Ethambutol, Fosfomycin, Furazolidone, Isoniazid, Linezolid, Metronidazole, Nitrofurantoin, Pyrazinamide, Quinupristin/Dalfopristin, Rifampin, and Spectinomycin.

The anti-fungal antibiotic agent may be of the polyene type and may be selected from the group consisting of Nystatin, Amphotericin B and Natamycin. In one aspect, the anti-fungal agent is Nystatin.

Patients being treated with chemotherapy and radiation are immuno-compromised. This leads to not only increased risk of bacterial infection but also to an increased risk of fungal infection, thus advancing the risk and degree of oral mucositis. Nystatin acts to prevent and limit the degree of fungal infection.

Alternatively, the anti-fungal compound may be selected from an imidazole, for example, Miconazole, Ketoconazole, Clotrimazole, Econazole, Mebendazole, Bifonazole, Butoconazole, Fenticonazole, Isoconazole, Oxiconazole, Sertaconazole, Sulconazole, Thiabendazole or Tiaconazole; a triazole, for example, Fluconazole, Itraconazole, Ravuconazole, Posaconazole or Voriconazole; an allylamine, for example, Terbenafine, Amorolfine, Naftifine or Butenafine or an echinocandin such as Caspofungin or Micafungin or any combinations thereof.

The topical anaesthetic may be selected from Benzocaine, Mepivacaine, Ropivacaine, Bupivacaine, Lidocaine, Prilocalne, Procaine, Cloroprocaine or Tetracaine. In one aspect the topical anaesthetic is a combination of Lidocaine and Tetracaine.

Pain associated with oral mucositis can be extremely debilitating and lead to poor oral food intake. In extreme cases patients may require feeding tubes if the ulceration continues to advance. Tetracaine and Lidocaine act locally to provide relief from pain.

Preferably, the anti-histamine may be selected from the group comprising a first generation H1 receptor antagonist, a second generation H1 receptor antagonist, a third generation H1 receptor antagonist, a H2 receptor antagonist, a H3 receptor antagonist and a H4 receptor antagonist.

Diphenyhdramine, an antihistamine, helps to sooth soreness, burning, itching (urticaria and pruritis) and inflammation, symptoms that are normally associate with mucositis.

The first generation H1 receptor antagonist may be selected from an ethylenediamine, for example, Mepyramine or Antazoline; an ethanolamine, for example, Diphenhydramine, Carbinoxamine, Doxylamine, Clemastine or Dimenhydrinate; an alkylamine, for example, Pheniramine, Chlorphenamine, Dexchlorphenamine, Brompheniramine or Triprolidine; a piperazine, for example, Cyclizine, Hydroxyzine, Meclizine or a tricyclic, for example, Promethazine, Alimemazine, Cyproheptadine or Azatadine. In one aspect, the anti-histamine is Diphenhydramine.

The second generation H1 receptor antagonist may be Azelastine, Levocabastine or Olopatadine.

The H3 receptor antagonist may be Thioperamide, Clobenpropit or Impromidine.

The H4 receptor antagonist may be Thioperamide.

Preferably, Dexamethasone is used in the range of from 0.005 mg/ml to 0.025 mg/ml, or, more preferably, narrower ranges such as 0.01 mg/ml to 0.02 mg/ml or 0.015 mg/ml to 0.02 mg/ml.

Preferably, Diphenhydramine is used in a range of from 1 mg/ml to 2 mg/ml or, more preferably, in narrower ranges such as 1 mg/ml to 1.5 mg/ml, 1.2 mg/ml to 1.4 mg/ml, or 1.25 mg/ml to 1.3 mg/ml.

Preferably, Lidocaine is used in a range of from 0.1% to 1% or, more preferably, in narrower ranges such as 0.2% to 0.9%, 0.3% to 0.8%, 0.4% to 0.7%, or 0.5% to 0.6%.

Preferably, Nystatin is used in an amount greater than 10,000 iu/ml. More preferably, Nystatin is used in a range of 10,000 iu/ml to 50,000 iu/ml, or 20,000 iu/ml to 40,000 iu/ml, or 30,000 iu/ml to 35,000 iu/ml, or most preferably at a concentration of 33,333 iu/ml.

Preferably, Erythromycin is used in a range of from 5 mg/ml to 15 mg/ml, or, more preferably, in narrower ranges such as 10 mg/ml to 15 mg/ml, 12 mg/ml to 15 mg/ml, or 12.5 mg/ml to 15 mg/ml.

In a further embodiment, the present invention provides a mouthwash comprising 0.015 mg/ml dexamethasone, 1.25 mg/ml diphenhydramine, 0.5% Lidocaine, 20000 iu/ml Nystatin, 12.5 mg/ml Erythromycin, polysorbate 20, spearmint oil, sorbitol, xanthan gum, sodium benzoate, purified water, aluminium hydroxide and magnesium hydroxide.

In a further aspect the present invention provides a method of treating or preventing oral conditions such as mucositis or stomatitis comprising rinsing the oral cavity with the mouthwash of the invention.

The present invention also provides, in a further embodiment, a kit for the treatment or prophylaxis of mucositis or stomatitis comprising a container having an amount of mouthwash in accordance with the present invention together with a set of instructions for using the mouthwash. The kit may optionally provide a cup or other container for conveniently dispensing an amount of the mouthwash from the container. Advantageously the cup may also have markings or other indicators for the convenient dispense of a measurement of a therapeutically effective dose of the mouthwash.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. The disclosures of all prior art recited herein are incorporated herein by reference in their entirety.

We claim:

1. An oral rinse formulation comprising one or more of the following components in combination with pharmaceutically acceptable excipients or additives:
a) at least one corticosteroid;
b) at least one anti-histamine;
c) at least one topical anaesthetic;
d) at least one antibiotic agent; and,
e) at least one anti-fungal antibiotic agent.

2. The formulation of claim 1 wherein all of components (a) to (e) are included.

3. The formulation of claim 1 having the following composition: 0.015 mg/ml dexamethasone; 1.25 mg/ml diphenhydramine; 0.5% Lidocaine; 20000 iu/ml Nystatin; and, 12.5 mg/ml Erythromycin.

4. The formulation of claim 3 further comprising polysorbate 20, spearmint oil, sorbitol, xanthan gum, sodium benzoate, purified water, aluminium hydroxide and magnesium hydroxide.

5. Use of the formulation of claim 1 for the treatment of oral lesions.

6. The use of claim 5 for the treatment of mucositis or stomatitis

7. A method of treating or preventing mucositis or stomatitis comprising applying to a patient an oral rinse formulation comprising one or more of the following components in combination with pharmaceutically acceptable excipients or additives:
a) at least one corticosteroid;
b) at least one anti-histamine;
c) at least one topical anaesthetic;
d) at least one antibiotic agent; and,
e) at least one anti-fungal antibiotic agent.
